(19) United States
(12) Patent Application Publication
(10) Pub. No.: US 2004/0243238 A1
(43) Pub. Date: Dec. 2, 2004

(54) Spinal disc prosthesis

(76) Inventors: Uri Arnin, Kiryat Tivon (IL); Yuri Sudin, Lod (IL); Michael Tauber, Tel Aviv (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/735,646

(22) Filed: Dec. 16, 2003

Related U.S. Application Data
(60) Provisional application No. 60/512,194, filed on Oct. 20, 2003.
(60) Provisional application No. 60/474,613, filed on Jun. 2, 2003.

(51) Int. Cl. A61F 2/44

(52) U.S. Cl. 623/17.12; 623/17.13

(57) ABSTRACT

A spinal disc prosthesis including an outer case adapted for attachment to spinal structure of a body, a cartridge removably mounted in the outer case, and a shock absorbing core within the cartridge.

### CROSS-REFERENCE TO RELATED APPLICATIONS

This application claims priority under 35 U.S.C §119 to U.S. Provisional Patent Application Serial No. 60/512,194, filed on Oct. 20, 2003, and U.S. Provisional Patent Application Serial No. 60/474,613, filed on Jun. 2, 2003, all of which are incorporated herein by reference.

### FIELD OF THE INVENTION

The present invention relates to human prostheses, and more particularly to spinal column vertebral disc prostheses.

### BACKGROUND OF THE INVENTION

The herniation of a spinal disc and the often resultant symptoms of intractable pain, weakness, sensory loss, incontinence and progressive arthritis are among the most common of debilitating processes affecting mankind. If a patient's condition does not improve after conservative treatment, and if clear physical evidence of nerve root or spinal cord compression is apparent, and if correlating radiographic studies (i.e., MRI or CT imaging or myelography) confirm the condition, surgical removal of the herniated disc may be indicated. The process of discectomy—as the name implies—involves the simple removal of the disc without attempt to replace or repair the malfunctioning unit.

Statistics suggest that present surgical techniques are likely to result in short-term relief, but will not prevent the progressive deterioration of the patient's condition in the long run. Through better pre-operative procedures and diagnostic studies, long-term patient results have improved somewhat. But it has become clear that unless the removed disc is replaced or the spine is otherwise properly supported, further degeneration of the patient's condition will almost certainly occur.

### SUMMARY OF THE INVENTION

The present invention seeks to provide a novel spinal prosthesis, as is described more in detail hereinbelow.

There is thus provided in accordance with an embodiment of the present invention a spinal disc prosthesis including an outer case adapted for attachment to spinal structure of a body, a cartridge removably mounted in the outer case, and a shock absorbing core within the cartridge.

The spinal disc prosthesis can include one or more of the following features. For example, the cartridge can be releasably locked to the outer case. An outer surface of the outer case can be coated with a semi-porous, bone-growth-encouraging material. The outer surface of the outer case can include an anchoring element. The shock absorbing core can include an elastomer material and/or a fluid shock-absorbing material. The shock absorbing core can include materials with different resilient properties. The shock absorbing core can be at least partially surrounded by an elastomeric ring.

### BRIEF DESCRIPTION OF THE DRAWINGS

In the drawings:

FIGS. 1 and 2 are cross-sectional and exploded views, respectively, of a spinal disc prosthesis, constructed and operative in accordance with an embodiment of the present invention.

### DESCRIPTION OF EMBODIMENTS

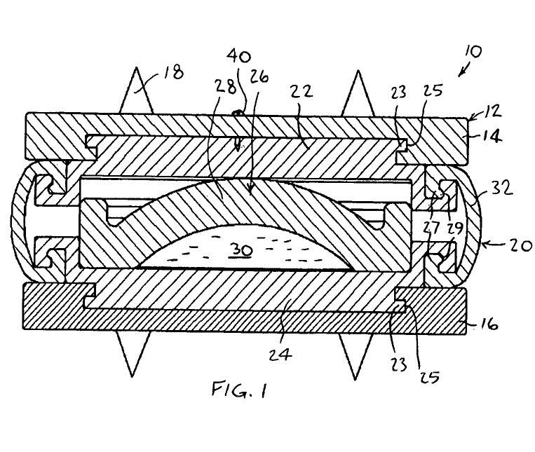

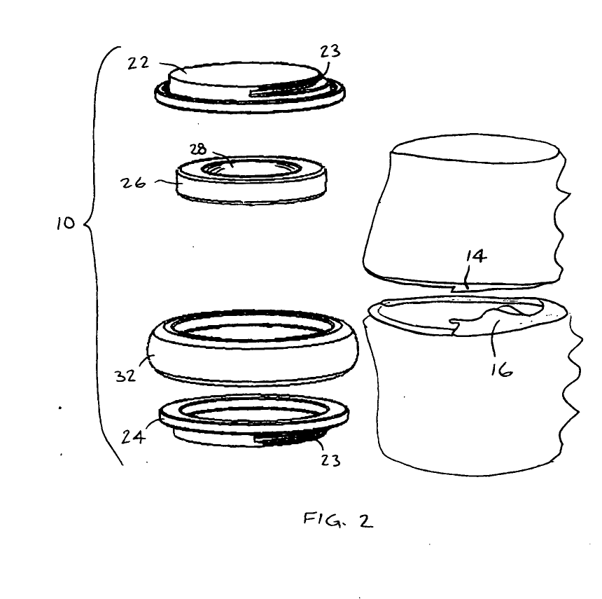

Reference is now made to FIGS. 1 and 2, which illustrate a spinal disc prosthesis 10, constructed and operative in accordance with an embodiment of the present invention.

Prosthesis 10 may comprise an outer case 12 adapted for attachment to spinal structure of a body, such as but not limited to, the vertebrate. Outer case 12 may include an upper plate 14 and a lower plate 16 (i.e., upper and lower in the sense of the drawing), which may be made of medically-safe metal or plastic. The outer surfaces of plates 14 and 16 may be attached to spinal structure of a body by any convenient method. The outer surfaces of plates 14 and 16 may be coated with a material, such as but not limited to, Hydroxy-Appatite (H/A), which encourages bone growth into the outer surfaces. Additionally or alternatively, an anchoring element 18 such as dorsal and ventral teeth may be provided to anchor plates 14 and 16 into the adjacent vertebrate bone structures.

A cartridge 20 may be removably mounted in outer case 12. Cartridge 20 may be constructed of a metal or plastic housing, comprising upper and lower housing halves 22 and 24, respectively. Upper and lower housing halves 22 and 24 may be slidingly attached to upper and lower plates 14 and 16, respectively, by means of a mortise-and-tenon joint, that is, tenons (tongues or other protrusions) that mate with corresponding mortises (grooves or recesses). For example, tenons 23 may be included on upper and lower housing halves 22 and 24 that slide or snap into mortises 25 formed in upper and lower plates 14 and 16. Alternatively, tenons 23 may be included on upper and lower plates 14 and 16 that slide or snap into mortises 25 formed in upper and lower housing halves 22 and 24. It is emphasized that this is just one example of a removable connection between cartridge 20 and outer case 12, and other kinds of attachments may be employed.

Cartridge 20 may include a shock absorbing core 26. Shock absorbing core 26 may be made of an elastomeric material 28, such as but not limited to, natural or synthetic rubber, which is configured to deform, shift, compress and the like to provide damping and shock absorption. Shock absorbing core 26 may have an arch/dome-like shape, such as that of a Belleville washer. Alternatively or additionally, the shock absorbing core 26 may include springs and dampers. Additionally or alternatively, shock absorbing core 26 may be constructed of a fluid shock-absorbing material 30, at least in part, such as but not limited to, a hydrogel that may become hydrated with time. Alternatively, shock absorbing core 26 may have a hollow chamber that can be at least partially filled with the fluid shock-absorbing material 30, which may become hydrated with time.

The shock absorbing core 26 may be surrounded (completely or partially) by an elastomeric ring 32, which may be fixed to upper and lower housing halves 22 and 24 by means of a mortise-and-tenon joint or any other attachment method, such as but not limited to, bonding with an adhesive. For example, tenons 27 may be included on elastomeric ring 32 that slide or snap into mortises 29 formed in upper and lower housing halves 22 and 24. Alternatively, tenons 27 may be included on upper and lower housing halves 22 and 24 that slide or snap into mortises 29 formed in elastomeric ring 32. It is emphasized that this is just one example of a removable connection between elastomeric ring 32 and on upper and lower housing halves 22 and 24, and other kinds of attachments may be employed. Elastomeric ring 32 permits and does not interfere with flexure of the shock absorbing core 26.

Shock absorbing core 26 may be of a uniform consistency or alternatively may comprise numerous sections with different resilient properties, e.g., made of different types of elastomers.

Cartridge 20 may be releasably locked to outer case 12. For example, a locking mechanism 40 may be provided that reversibly attaches cartridge 20 to upper and/or lower plates 14 and 16. Locking mechanism 40 may include, without limitation, a screw, clasp or other mechanical fastener, for example.

Cartridge 20 can be inserted into upper and lower housing halves 14 and 16 and/or replaced in case of necessity. The core 26 may require replacement, for example, if material deterioration occurs. This can be easily accomplished by releasing locking mechanism 40 (if provided), and then removing cartridge 20 by simply sliding it out of outer case 12. A replacement cartridge 20 can be immediately substituted and locked into position, thus saving any intricate procedures as described in the introduction.

Many alternatives, modifications and variations will be apparent to those skilled in the art. Accordingly, all such alternatives, modifications and variations that fall within the spirit and broad scope of the appended claims.

What is claimed is:

1. A spinal disc prosthesis comprising:
an outer case adapted for attachment to spinal structure of a body;
a cartridge removably mounted in said outer case; and
a shock absorbing core within the cartridge.

2. The spinal disc prosthesis according to claim 1, wherein said cartridge is releasably locked to said outer case.

3. The spinal disc prosthesis according to claim 1, wherein an outer surface of said outer case is coated with a bone-growth-encouraging material.

4. The spinal disc prosthesis according to claim 1, wherein an outer surface of said outer case comprises at least one anchoring element.

5. The spinal disc prosthesis according to claim 1, wherein said shock absorbing core comprises an elastomer material.

6. The spinal disc prosthesis according to claim 1, wherein said shock absorbing core comprises a fluid shock-absorbing material.

7. The spinal disc prosthesis according to claim 1, wherein said shock absorbing core comprises materials with different resilient properties.

8. The spinal disc prosthesis according to claim 1, wherein said shock absorbing core is at least partially surrounded by an elastomeric ring.

9. The spinal disc prosthesis according to claim 1, wherein said outer case comprises an upper plate and a lower plate attachable to spinal structure of a body.
